(19) United States
(12) Patent Application Publication
(10) Pub. No.: US 2010/0049061 A1
(43) Pub. Date: Feb. 25, 2010

(54) SYSTEMS AND METHODS FOR PROVIDING AUDITORY FEEDBACK DURING CATHETER PLACEMENT

(75) Inventors: Daniel J. Wilson, Sammamish, WA (US); Diane S. Paine, Redmond, WA (US)

Correspondence Address: STOEL RIVES LLP - SLC, 201 SOUTH MAIN STREET, SUITE 1100, ONE UTAH CENTER, SALT LAKE CITY, UT, 84111, US

(73) Assignee: MR HOLDINGS (HK) LTD., Hong Kong (CN)

(21) Appl. No.: 12/544,829

(22) Filed: Aug. 20, 2009

Related U.S. Application Data
(60) Provisional application No. 61/090,804, filed on Aug. 21, 2008.

(51) Int. Cl. A61B 5/0215

(52) U.S. Cl. 600/486

(57) ABSTRACT

A system for providing auditory feedback may include a blood pressure monitor interface for acquiring pressure waveform data from a pressure transducer indicative of blood pressure readings over time. The system may further include an anatomic location annunciator for generating an audible signal from the pressure waveform data that conveys information about the anatomic location of the pressure transducer within the heart chamber or blood vessel.

### CROSS-REFERENCE TO RELATED APPLICATIONS

This application claims the benefit of U.S. Provisional Application No. 61/090,804, filed Aug. 21, 2008, which is incorporated herein by reference.

### TECHNICAL FIELD

This disclosure relates generally to invasive blood pressure (IBP) monitoring and, more particularly, to techniques for converting IBP readings into auditory feedback to assist in catheter placement.

### BRIEF DESCRIPTION OF THE DRAWINGS

FIG. 1 is block diagram for system for providing auditory feedback during catheter placement;

FIG. 2 is a block diagram of an anatomic location annunciator including a tone generator;

FIG. 3 is a block diagram of an anatomic location annunciator including a pattern matcher;

FIG. 4 illustrates various pressure waveform characteristics that can be identified by the pattern matcher of FIG. 3;

FIG. 5 illustrates a pattern matcher that accepts as input both IBP and ECG (electrocardiogram) waveform data;

FIG. 6 is a block diagram of an anatomic location annunciator including a signature generator for adding a new pressure waveform signature to a signature library;

FIG. 7 is a flowchart of a method for providing auditory feedback during catheter placement; and

FIG. 8 is a flowchart of a method for adding a new pressure waveform signature to a signature library.

### DETAILED DESCRIPTION

When catheters, such as pulmonary artery catheters, are placed into patients, imaging is typically not available to allow a clinician to visualize the catheter location at the time of insertion. Clinicians are forced to rely upon their knowledge of typical pressure waveform characteristics and pressure magnitudes in blood vessels and chambers of the heart in order to identify the anatomic location of the catheter. However, when inserting an indwelling catheter, the clinician's focus of attention is the catheter insertion site and the insertion procedure. Conventionally, determining the pressure waveform requires the clinician to look away from the insertion site (and the patient) to observe the invasive blood pressure (IBP) parameter of the monitoring system, thus dividing the clinician's attention and increasing the likelihood of error.

The present disclosure relates to systems and methods for providing auditory feedback to eliminate the need for the clinician to watch an IBP monitor during catheter placement. One aspect of the disclosure includes a method for providing auditory feedback about the anatomic location of a pressure transducer coupled to a catheter during insertion thereof into a heart chamber or blood vessel of a patient. The method may include acquiring pressure waveform data from the pressure transducer indicative of blood pressure readings over time. The method may further include generating an audible signal from the pressure waveform data that conveys information about the anatomic location of the pressure transducer within the heart chamber or blood vessel.

In one embodiment, the audible signal is a tone, and the process of generating the audible signal may include modulating the pitch of the tone according to the pressure waveform data. Higher pitched tones may represent higher blood pressure readings in the pressure waveform data, and lower pitched tones may represent lower blood pressure readings in the pressure waveform data.

In another embodiment, the process of generating the audible signal may include matching the acquired pressure waveform data with a pressure waveform signature for particular location within the heart chamber or blood vessel. The process may further include locating a stored audio signal associated with the matching pressure waveform signature and outputting the stored audio signal to the clinician. In one embodiment, the stored audio signal may be a prerecorded voice prompt describing the anatomic location of the pressure transducer.

The pressure waveform signature may include a representation of typical pressure waveform data for the particular location. Alternatively, the pressure waveform signature may include a stored representation of pressure waveform data previously acquired at the particular location.

In certain embodiments, the process of matching the pressure waveform data may include determining one or more pressure waveform characteristics of the pressure waveform data and locating a pressure waveform signature having the one or more pressure waveform characteristics. Various waveform characteristics that may be identified include, but are not limited to:
a biphasic waveform with a systolic component and a diastolic component;
a magnitude of pulse pressure determined by subtracting diastolic pressure from systolic pressure;
a dicrotic notch at an end-systolic portion of a pressure waveform;
a continual increase or decrease in diastolic pressure; and
timing of pressure waveform data relative to ECG waveform data.

An aspect of the present disclosure includes a method for adding pressure waveform signatures to a signature library. The method may include storing a pressure waveform signature corresponding to pressure waveform data acquired at a new location of the pressure transducer. The method may also include recording an audio signal comprising a voice prompt describing the anatomic location of the pressure transducer. The method may further include associating the recorded audio signal with the stored pressure waveform signature, such that a subsequent acquisition of similar pressure waveform data by the pressure transducer will trigger annunciation of the voice prompt.

Another aspect of the present disclosure includes a system for providing auditory feedback about the anatomic location of a pressure transducer coupled to a catheter during insertion thereof into a heart chamber or blood vessel of a patient. The system may include a blood pressure monitor interface for acquiring pressure waveform data from a pressure transducer indicative of blood pressure readings over time. The system may further include an anatomic location annunciator for generating an audible signal from the pressure waveform data that conveys information about the anatomic location of the pressure transducer within the heart chamber or blood vessel.

In one embodiment, the anatomic location annunciator may include a tone generator for modulating the pitch of a tone according to the pressure waveform data. Alternatively, or in addition, the anatomic location annunciator may include a pattern matcher for matching the pressure waveform data with a pressure waveform signature for a particular location within the heart chamber or blood vessel and locating a stored audio signal associated with the matching pressure waveform signature. The anatomic location annunciator may further include an audio player to output the stored audio signal to a clinician.

In one embodiment, the anatomic location annunciator may include or have access to a signature library comprising a plurality of pressure waveform signatures corresponding to different locations within the heart chamber or blood vessel. The pressure waveform signatures within the signature library may comprise typical pressure waveform data for the particular location or may have been previously acquired at the particular location.

In one embodiment, the system may include a signature generator for generating and storing a pressure waveform signature corresponding to the pressure waveform data acquired at the anatomic location of the pressure transducer. The system may further include a voice digitizer for recording an audio signal comprising a voice prompt describing the anatomic location of the pressure transducer within the heart chamber or blood vessel. The voice digitizer may store the recorded voice prompt within the signature library, such that a subsequent acquisition of similar pressure waveform data by the pressure transducer will trigger annunciation of the voice prompt.

The embodiments of the disclosure will be best understood by reference to the drawings, wherein like elements are designated by like numerals throughout. In the following description, numerous specific details are provided for a thorough understanding of the embodiments described herein. However, a skilled artisan will recognize that one or more of the specific details may be omitted, or other methods, components, or materials may be used. In some cases, operations are not shown or described in detail.

The described features, operations, or characteristics may be combined in any suitable manner in one or more embodiments. It will also be readily understood that the order of the steps or actions of the methods described in connection with the disclosed embodiments may be changed as would be apparent to a skilled artisan. Thus, any order in the drawings or Detailed Description is for illustrative purposes only and is not meant to imply a required order, unless specified to require an order.

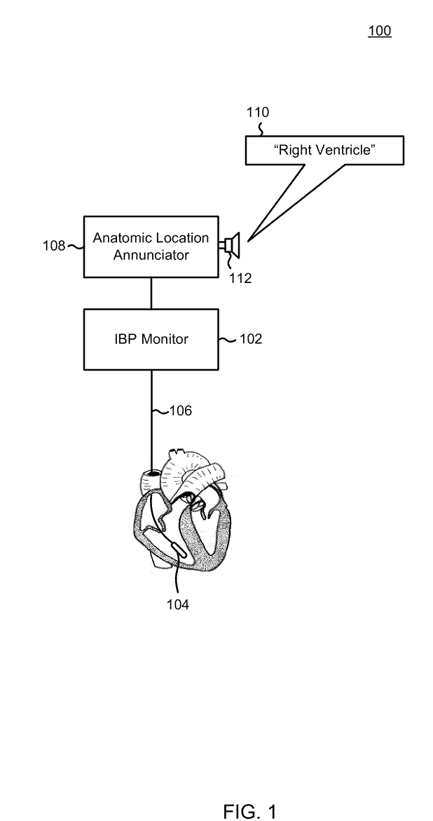

FIG. 1 is a block diagram of a system 100 for providing auditory feedback during catheter placement. In one embodiment, an invasive blood pressure (IBP) monitor 102 receives pressure waveform data from a pressure transducer 104 coupled to a catheter 106 during insertion thereof into a blood vessel or chamber of the heart. The pressure transducer 104 converts mechanical pressure exerted by blood within the vessel or chamber into electrical signals that are detectable by the IBP monitor 102. The pressure waveform data represent blood pressure readings taken over time at a predetermined or user-defined sample frequency.

Various IBP monitors 102 are known in the art, such as the Mindray PM9000 Express patient monitor, available from Shenzhen Mindray Bio-Medical Electronics, Co., Ltd., of Shenzhen, China, which monitors a variety of physiological parameters, including IBP. The IBP monitor 102, as well as the other components of the system 100 described hereafter, may be embodied as any suitable combination of hardware, software, and/or firmware and may operate within the context of a general purpose computer including a processor, memory, and other standard components known to a skilled artisan.

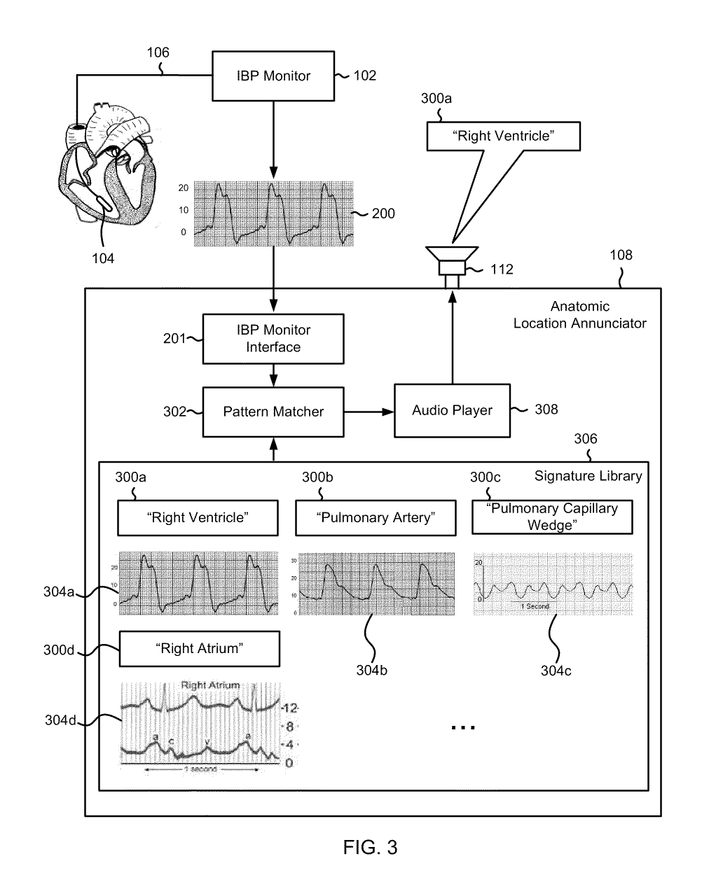

The IBP monitor 102 is coupled to or otherwise in communication with an anatomic location annunciator 108. The anatomic location annunciator 108 may be a component of the IBP monitor 102 or a separate software program or device that communicates with the IBP monitor 102 via wireless or wired communication protocols. The anatomic location annunciator 108 converts the pressure waveform data into auditory feedback 110 about the anatomic location of the pressure transducer 104 within the vessel or chamber, which may be output to the clinician by a speaker 112. As described in greater detail with reference to FIG. 3, the auditory feedback 110 may be a voice prompt (e.g., “right ventricle”) comprising a spoken description of the anatomic location of the pressure transducer 104.

By virtue of the auditory feedback 110 provided by the methods and systems disclosed herein, a clinician need not look away from the insertion site during catheter placement. This greatly reduces the risk of error because the clinician can devote his undivided attention to the insertion procedure.

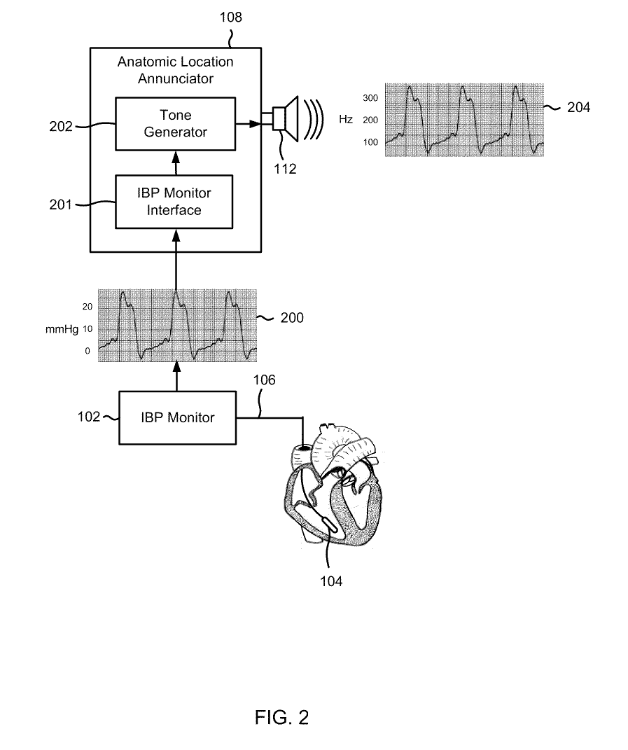

FIG. 2 provides additional details of the anatomic location annunciator 108 depicted in FIG. 1. As previously described, the IBP monitor 102 obtains pressure waveform data 200, which is typically represented in units of millimeters of mercury (mmHg) for each sample period over a time interval. The pressure waveform data 200 is provided or made accessible to the anatomic location annunciator 108 by the IBP monitor 102.

In one embodiment, the anatomic location annunciator 108 includes an IPB monitor interface 201 for communicating with the IPB monitor 102. The IPB monitor interface 201 may implement all of the necessary protocols for receiving the pressure waveform data 200 from the IPB monitor 102 and may vary depending on the particular IBP monitor 102 in use. The IPB monitor interface 201 may be implemented in software or using any suitable combination of hardware, software, and/or firmware, and may be provided by (or conform to specifications on the manufacturer of the IPB monitor 102.

The anatomic location annunciator 108 may further include a tone generator 202 for modulating the pitch (frequency) of a tone 204 according to the pressure waveform data 200. For example, the tone generator 202 may generate a higher pitched tone to represent a higher blood pressure reading in the pressure waveform data 200 and a lower pitched tone to represent a lower blood pressure reading in the pressure waveform data 200. Software-based tone generators 202 include the NCH Tone Generator, available from NCH Software. However, a skilled artisan will recognize that hardware-based tone generators 202 may also be used.

A clinician with knowledge of typical pressure waveforms for various blood vessels and chambers of the heart will be able to interpret the modulated pitch as an indication of the anatomic location of the pressure transducer 104. For example, the clinician may recognize that a constant tone pitch will be annunciated when monophasic pressure waveforms are present, indicating that the pressure transducer 104 might be in the right atrium of the heart. Likewise, the clinician may recognize that a changing tone pitch will be annunciated when biphasic pressure waveforms are present, indicating that the pressure transducer 104 might be in the right ventricle or pulmonary artery. While such an approach relies on the skill of the clinician in recognizing pressure waveforms, it is superior to conventional techniques that require the clinician to look away from the insertion site.

In one embodiment, the frequency of the tone 204 may be derived from the pressure waveform data 200 using the following equation:

f=s(p+t) Eq. 1

where:
f is the frequency of the tone in Hz;
s is a scaling factor (e.g., 10);
p is a pressure reading in mmHg; and
t is a transposition (e.g., 10).
   An artisan will recognize that various formulas may be used to calculate the frequency of the tone 204. Moreover, the formula may vary according to user preferences. For instance, a user may prefer to have higher or lower pitched tones or a greater or lesser degree of variation between minimum and maximum pitch. This may be accomplished by modifying the scaling and transposition parameters, preferably within a range to produce an audible signal that is reproducible by the speaker 112.

FIG. 3 illustrates an alternative embodiment of the anatomic location annunciator 108 that produces auditory feedback in the form of a voice prompt 300 (four depicted as 300a-d). In one implementation, the voice prompt 300 is a recorded audio signal in which the anatomic location of the pressure transducer 104 is audibly spoken (e.g., “right ventricle”).

The anatomic location annunciator 108 may include a pattern matcher 302 that receives the pressure waveform data 200 from the IPB monitor interface 201. As described in greater detail below, the pattern matcher 302 compares acquired pressure waveform data 200 (or characteristics thereof) with a plurality of stored pressure waveform signatures 304 (four shown as 304a-d) to determine if a match is found. The pressure waveform signatures 304a-d may be stored within a signature library 306 that associates pressure waveform signatures 304a-d with stored voice prompts 300a-d. The voice prompt 304a associated with the matching pressure waveform signature 304a (e.g., “right ventricle”) is sent to an audio player 308 to be output via the speaker 112.

Pattern matching is known in the art of speech recognition, which correlates speech waveforms with stored signatures for various words or phrases. Hence, one embodiment of the pattern matcher 302 may be implemented using speech recognition algorithms, such as algorithms based on Hidden Markov Models (HMMs). Commercially available speech recognition programs that may be adapted to recognize pressure waveform data 200 include the Dragon Naturally Speaking SDK 9 available from Nuance Communications, Inc.

The pressure waveform signatures 304a-d may comprise actual pressure waveform data 200 previously monitored within particular heart chambers or blood vessels of one or more patients (including the patient being currently monitored). Alternatively, or in addition, the pressure waveform signatures 304a-d may reflect typical pressure waveforms known to be associated with particular heart chambers or blood vessels. In other words, the pressure waveform signatures 304a-d need not contain data that has actually been obtained from a patient, but may be based on previous studies of pressure waveforms within normal and abnormal human circulatory systems.

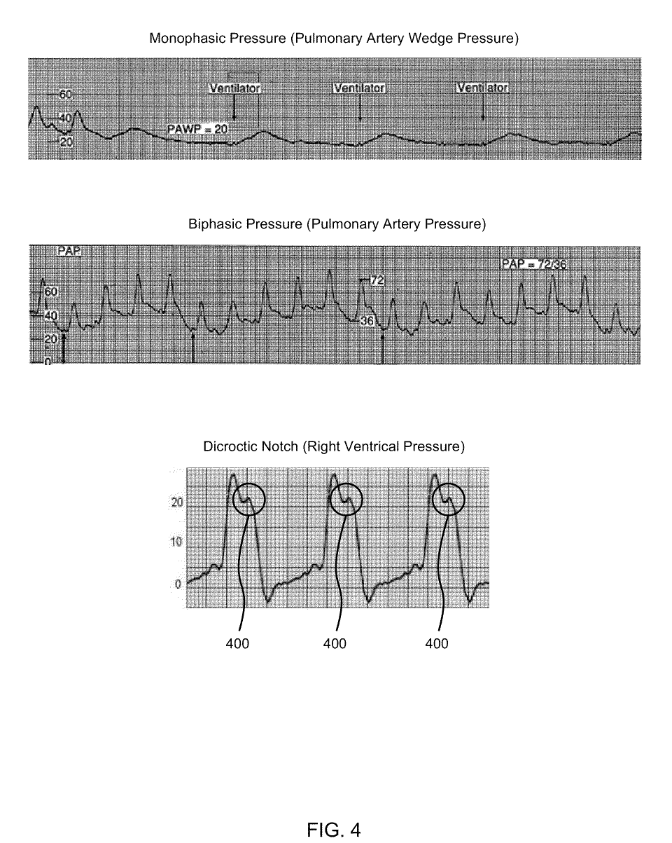

In certain embodiments, the pressure waveform signatures 304 may not include pressure waveform data 200 in the form of pressure readings over time, but, rather, representations of such data in the form of polynomial curves, features, characteristics, heuristics, rules, or the like. Such an embodiment will be described in greater detail in connection with FIG. 4

Voice prompts 304a-d may be stored, for example, as pulse-code modulated (PCM) or MPEG Layer 3 (MP3) audio data. The audio player 308 may be implemented using any suitable software program or device for decoding and outputting audio signals. For instance, the audio player 308 may be implemented by Windows Media Player available from Microsoft Corporation.

The signature library 306 that associates the voice prompts 300a-d with the corresponding pressure waveform signatures 304a-d may be implemented within the context of a relational database management system (RDBMS), such as the Oracle RDBMS, available from Oracle Corporation, or DB2, available from IBM. Although the signature library 306 is depicted as being within the anatomic location annunciator 108, a skilled artisan will recognize that the signature library 306 may be stored remotely and accessed using client software (not shown) over a network.

In some embodiments, the anatomic location annunciator 308 may include both the tone generator 202 of FIG. 2 and the pattern matcher 302 of FIG. 3, allowing the user to select between tone and voice annunciation. Selection may be accomplished by a hardware- or software-based control (not shown) accessible to the clinician. Certain embodiments may present both tone and voice annunciation at the same time.

Referring to FIG. 4, the pattern matcher 302, alternatively or in addition, may look for various pressure waveform characteristics in the pressure waveform data 200 indicative of particular blood vessels or chambers of the heart. FIG. 4 illustrates examples of identifiable waveform characteristics, such as monophasic pressure, biphasic pressure, and a dicrotic notch 400.

Monophasic pressure, which is characterized by the lack of systolic/diastolic fluctuations, may be indicative of pulmonary artery wedge pressure. If detected, the pattern matcher 302 may annunciate the anatomic location as the pulmonary capillary wedge. By contrast, strong systolic and diastolic fluctuations, as well as the magnitude of the pulse pressure (systolic minus diastolic pressure), may be used by the pattern matcher 302 to recognize the biphasic pressure typically found in the pulmonary artery.

Identification of a dicrotic notch 400 on the end systolic portion of a pressure waveform may be indicative of right ventricle pressure. The dicrotic notch 400 may be identified as a sign change in the second derivative of the pressure waveform shortly after detection of the maximum systolic pressure. By contrast, the lack of a dicrotic notch, with the waveform reflecting a continual increase or decrease in diastolic pressure, may indicate pulmonary artery pressure.

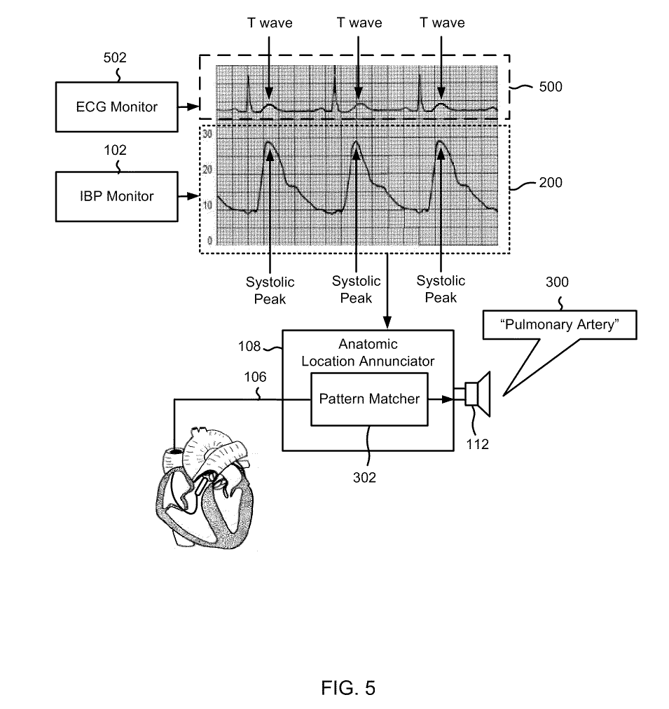

As illustrated in FIG. 5, the pattern matcher 302 may also receive ECG waveform data 500 from an ECG monitor 502. The timing of the pressure waveform data 200 relative to the diastolic and systolic portion of the ECG waveform data 502 may assist with identifying the anatomic location of the pressure transducer 104. For example, the systolic peak of pulmonary artery pressure occurs during the T wave of the ECG waveform data 500. Rules or other data for using ECG waveform data 502 in combination with the pressure waveform data 200 may be associated with the pressure waveform signatures 304 in one embodiment. A skilled artisan, with access to the present disclosure, will recognize that the pattern matcher 302 may be configured to detect other waveform characteristics known in the art.

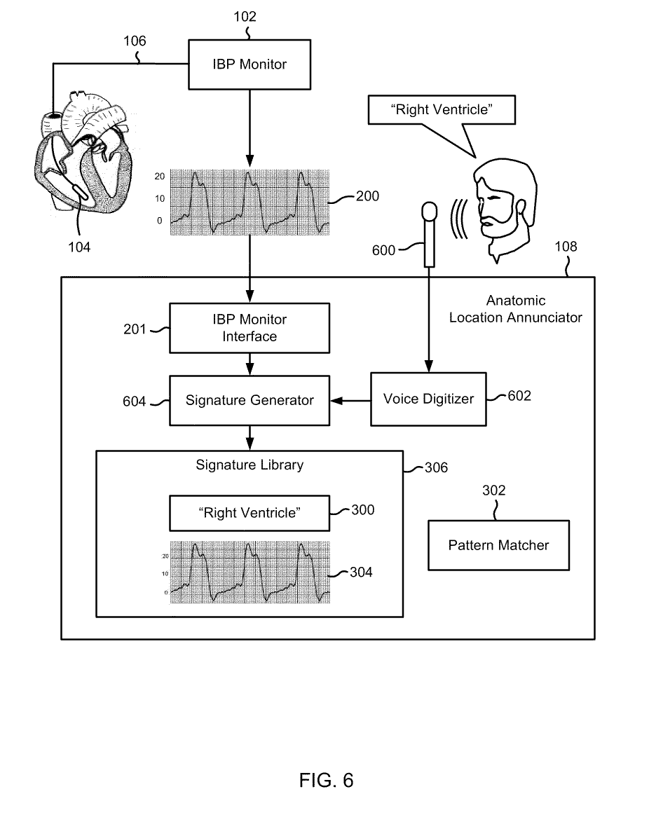

FIG. 6 illustrates an embodiment of the present disclosure in which a clinician may add new pressure waveform signatures 304 and associated voice prompts 300 to the signature library 306. As before, the IBP monitor 102 may obtain pressure waveform data 200. However, in some cases, no match may be found by the pattern matcher 302. For instance, the signature library 306 may be empty or the pressure waveform data 200 may reflect abnormal blood pressure readings due to a medical condition.

The clinician may choose (or be prompted) to record a voice prompt 300. The voice prompt 304 may be recorded via a microphone 602 coupled to a voice digitizer 602, such as Microsoft's Sound Recorder. The recorded voice prompt 300 and the pressure waveform data 200 received contemporaneously therewith is provided to a signature generator 604. In one embodiment, the signature generator 604 may store the pressure waveform data 200 as the pressure waveform signature 304. In other embodiments, the signature generator 604 may convert the pressure waveform data 200 into different representations, such as polynomial curves, features, characteristics, heuristics, or rules.

Once stored in the signature library 306, the pressure waveform signature 304 will enable the anatomic location annunciator 108 (and more particularly, the pattern matcher 302) to recognize the pressure waveform data 200 when it is subsequently monitored, allowing the associated voice prompt 300 to be output to the clinician.

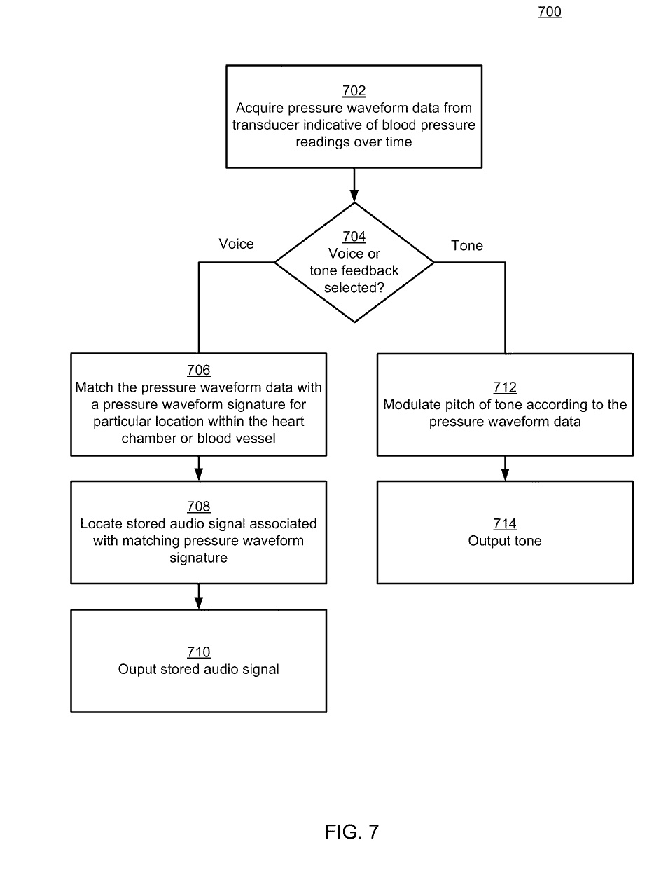

FIG. 7 is a flowchart of a method 700 for providing auditory feedback during catheter placement. Pressure waveform data, indicative of blood pressure readings over time, are acquired 702 from a pressure transducer coupled to a catheter. A determination 704 is made whether voice or tone feedback has been selected. If voice feedback has been selected, the pressure waveform data is matched 706 with a pressure waveform signature for a particular location within a heart chamber or blood vessel. A stored audio signal associated with the matching pressure waveform signature is located 708, after which the stored audio signal is output 710 to the clinician.

If, on the other hand, tone feedback has been selected, the pitch of a tone is modulated according to the pressure waveform data. For example, higher pitched tones may be generated to represent higher blood pressure readings in the pressure waveform data, and lower pitched tones may be generated to represent lower blood pressure readings in the pressure waveform data. The modulated tone is then output 714 to the clinician.

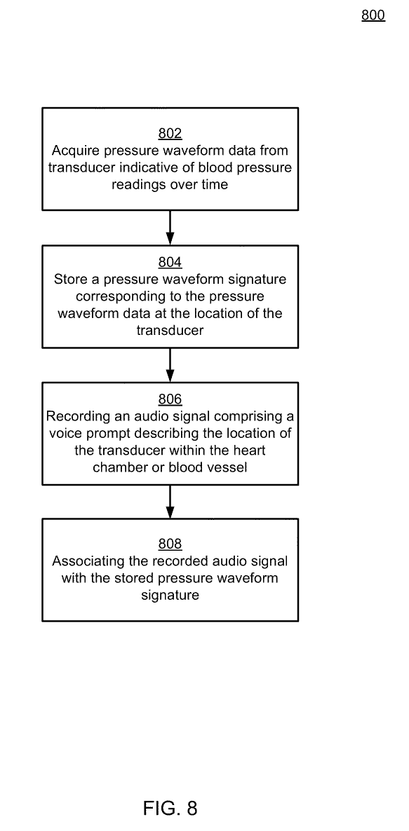

FIG. 8 is a flowchart of a method 800 for adding a pressure waveform signature to a signature library. Pressure waveform data is acquired 802 from the pressure transducer indicative of blood pressure readings over time. A pressure waveform signature is stored 804 corresponding to the pressure data at the anatomic location of the pressure transducer. An audio signal 806 is recoded comprising a voice prompt describing the anatomic location of the pressure transducer within the heart chamber or blood vessel, which is associated 808 with the stored pressure waveform signature.

Embodiments of the foregoing disclosure may include various steps, which may be embodied in computer-executable instructions to be executed by a general-purpose or special-purpose computer (or other electronic device). Alternatively, the steps may be performed by hardware components that include specific logic for performing the steps or by any suitable combination of hardware, software, and/or firmware.

Embodiments may also be provided as a computer program product including a computer-readable medium having stored thereon instructions that may be used to program a computer (or other electronic device) to perform processes described herein. The computer-readable medium may include, but is not limited to, hard drives, floppy diskettes, optical disks, CD-ROMs, DVD-ROMs, ROMs, RAMs, EPROMs, EEPROMs, magnetic or optical cards, solid-state memory devices, or other devices for storing electronic instructions.

Several aspects of the embodiments have been illustrated as software modules or components. As used herein, a software module or component may include any type of computer instruction or computer executable code located within a memory device and/or transmitted as electronic signals over a system bus or wired or wireless network. A software component may, for instance, comprise one or more physical or logical blocks of computer instructions, which may be organized as a routine, program, object, component, data structure, etc., that performs one or more tasks or implements particular abstract data types.

In certain embodiments, a particular software component may comprise disparate instructions stored in different locations of a memory device, which together implement the described functionality of the component. Indeed, a component may comprise a single instruction or many instructions, and may be distributed over several different code segments, among different programs, and across several memory devices. Some embodiments may be practiced in a distributed computing environment where tasks are performed by a remote processing device linked through a communications network. In a distributed computing environment, software components may be located in local and/or remote memory storage devices. In addition, data being tied or rendered together in a database record may be resident in the same memory device, or across several memory devices, and may be linked together in fields of a record in a database across a network.

Various modifications, changes, and variations apparent to those of skill in the art may be made in the arrangement, operation, and details of the methods and systems of the disclosure without departing from the spirit and scope of the disclosure. Thus, it is to be understood that the embodiments described above have been presented by way of example, and not limitation, and that the invention is defined by the appended claims.

What is claimed is:

1. A method for providing auditory feedback about the anatomic location of a pressure transducer coupled to a catheter during insertion thereof into a heart chamber or blood vessel of a patient, the method comprising:
acquiring pressure waveform data from the pressure transducer indicative of blood pressure readings over time; and
generating an audible signal from the pressure waveform data that conveys information about the anatomic location of the pressure transducer within the heart chamber or blood vessel.

2. The method of claim 1, wherein the audible signal comprises a tone, and wherein generating comprises:
modulating a pitch of the tone according to the pressure waveform data.

3. The method of claim 2, wherein a higher pitched tone represents a higher blood pressure reading in the pressure waveform data and a lower pitched tone represents a lower blood pressure reading in the pressure waveform data.

4. The method of claim 1, wherein generating comprises:
matching the pressure waveform data with a pressure waveform signature for particular location within the heart chamber or blood vessel;
locating a stored audio signal associated with the matching pressure waveform signature; and
outputting the stored audio signal.

5. The method of claim 4, wherein the pressure waveform signature comprises a stored representation of typical pressure waveform data for the particular location.

6. The method of claim 4, wherein the pressure waveform signature comprises a stored representation of pressure waveform data previously acquired at the particular location.

7. The method of claim 3, wherein matching comprises:
determining one or more pressure waveform characteristics of the pressure waveform data; and
locating a pressure waveform signature having the one or more pressure waveform characteristics.

8. The method of claim 7, wherein at least one pressure waveform characteristic comprises a biphasic waveform with a systolic component and a diastolic component.

9. The method of claim 7, wherein at least one pressure waveform characteristic comprises a magnitude of pulse pressure determined by subtracting diastolic pressure from systolic pressure.

10. The method of claim 7, wherein at least one pressure waveform characteristic comprises a dicrotic notch at an end-systolic portion of the pressure waveform data.

11. The method of claim 7, wherein at least one pressure waveform characteristic comprises a continual increase or decrease in diastolic pressure.

12. The method of claim 7, wherein at least one pressure waveform characteristic comprises timing of pressure waveform data relative to ECG waveform data.

13. The method of claim 4, wherein the stored audio signal comprises prerecorded voice prompt describing the anatomic location of the pressure transducer within the heart chamber or blood vessel.

14. The method of claim 4, further comprising:
storing a pressure waveform signature corresponding to pressure waveform data acquired by the pressure transducer at a new location within the heart chamber or blood vessel;
recording an audio signal comprising a voice prompt describing the new location; and
associating the recorded audio signal with the stored pressure waveform signature, such that a subsequent acquisition of similar pressure waveform data by the pressure transducer will trigger annunciation of the voice prompt.

15. A system for providing auditory feedback about the anatomic location of a pressure transducer coupled to a catheter during insertion thereof into a heart chamber or blood vessel of a patient, the system comprising:
a blood pressure monitor interface for acquiring pressure waveform data from the pressure transducer indicative of blood pressure readings over time; and
an anatomic location annunciator for generating an audible signal from the pressure waveform data that conveys information about the anatomic location of the pressure transducer within the heart chamber or blood vessel.

16. The system of claim 15, wherein the audible signal comprises a tone, and wherein the anatomic location annunciator comprises a tone generator for modulating a pitch of the tone according to the pressure waveform data.

17. The system of claim 16, wherein a higher pitched tone represents a higher blood pressure reading in the pressure waveform data and a lower pitched tone represents a lower blood pressure reading in the pressure waveform data.

18. The system of claim 15, wherein the anatomic location annunciator comprises:
a pattern matcher for matching the pressure waveform data with a pressure waveform signature for particular location within the heart chamber or blood vessel and locating a stored audio signal associated with the matching pressure waveform signature; and
an audio player for outputting the stored audio signal.

19. The system of claim 18, further comprising a signature library comprising a plurality of pressure waveform signatures corresponding to different locations within the heart chamber or blood vessel,

20. The system of claim 18, wherein the pressure waveform signature comprises a stored representation of typical pressure waveform data for the particular location.

21. The system of claim 18, wherein the pressure waveform signature comprises a stored representation of pressure waveform data previously acquired at the particular location.

22. The system of claim 18, wherein the pattern matcher is to determine one or more pressure waveform characteristics of the pressure waveform data and locate a pressure waveform signature having the one or more pressure waveform characteristics.

22. The system of claim 21, wherein at least one pressure waveform characteristic comprises a biphasic waveform with a systolic component and a diastolic component.

23. The system of claim 21, wherein at least one pressure waveform characteristic comprises a magnitude of pulse pressure determined by subtracting diastolic pressure from systolic pressure.

24. The system of claim 21, wherein at least one pressure waveform characteristic comprises a dicrotic notch at an end-systolic portion of the pressure waveform data.

25. The system of claim 21, wherein at least one pressure waveform characteristic comprises a continual increase or decrease in diastolic pressure.

26. The system of claim 21, wherein at least one pressure waveform characteristic comprises timing of pressure waveform data relative to ECG waveform data.

27. The system of claim 18, wherein the stored audio signal comprises prerecorded voice prompt describing the anatomic location of the pressure transducer within the heart chamber or blood vessel.

28. The system of claim 18, further comprising:
a signature generator for generating and storing a pressure waveform signature corresponding to pressure waveform data acquired by the pressure transducer at a new location within the heart chamber or blood vessel;
a voice digitizer for recording an audio signal comprising a voice prompt describing the anatomic location of the pressure transducer; and
a signature library for associating the recorded audio signal with the stored pressure waveform signature, such that a subsequent acquisition of similar pressure waveform data by the pressure transducer will trigger annunciation of the voice prompt.

28. The system of claim 15, wherein the anatomic location annunciator comprises:
a tone generator for modulating a pitch of a tone according to the pressure waveform data;
a pattern matcher for matching the pressure waveform data with a pressure waveform signature for particular location within the heart chamber or blood vessel and locating a stored audio signal associated with the matching pressure waveform signature; and
an audio output component for selectively outputting one or both of the tone or the stored audio signal.

29. An apparatus providing auditory feedback about the anatomic location of a pressure transducer coupled to a catheter during insertion thereof into a heart chamber or blood vessel of a patient, the apparatus comprising:
means for acquiring pressure waveform data from the pressure transducer indicative of blood pressure readings over time; and
means for generating an audible signal from the pressure waveform data that conveys information about the anatomic location of the pressure transducer within the heart chamber or blood vessel.

30. A computer-readable medium comprising program instructions for causing a computer to perform a method for providing auditory feedback about the anatomic location of a pressure transducer coupled to a catheter during insertion thereof into a heart chamber or blood vessel of a patient, the computer-readable medium comprising:
program instructions for acquiring pressure waveform data from the pressure transducer indicative of blood pressure readings over time; and
program instructions for generating an audible signal from the pressure waveform data that conveys information about the anatomic location of the pressure transducer within the heart chamber or blood vessel.
